(19) United States
(12) Patent Application Publication
(10) Pub. No.: US 2011/0184013 A1
(43) Pub. Date: Jul. 28, 2011

(54) Imidazo[1,2-a]Pyridine Compounds As Receptor Tyrosine Kinase Inhibitors

(76) Inventors: Shelley Allen, Loveland, CO (US); Fredrik P. Marmsäter, Boulder, CO (US); John E. Robinson, Commerce City, CO (US); Stephen T. Schlachter, Boulder, CO (US); Joseph P. Lyssikatos, Piedmont, CA (US)

(21) Appl. No.: 13/122,095

(22) PCT Filed: Sep. 30, 2009
(86) PCT No.: PCT/US09/59017

§ 371 Date: Mar. 31, 2011

Related U.S. Application Data
(60) Provisional application No. 61/101,984, filed on Oct. 1, 2008.

(51) Int. Cl. A61K 31/4709; C07D 471/04; A61P 35/00; A61P 43/00

(52) U.S. Cl. 514/300; 546/121

(57) ABSTRACT

Compounds of Formula I and II: I II having the chemical names cis-6-fluoro-8-(3-fluoropiperidin-4-yloxy)-2-(7-(2-methoxyethoxy)imidazo[1,2-a]pyridin-3-yl)quinoline and 6-fluoro-8-(trans-3-fluoropiperidin-4-yloxy)-2-(7-(2-methoxyethoxy)imidazo[1,2-a]pyridin-3-yl)quinoline, respectively, and enantiomers and pharmaceutically acceptable salts thereof, are receptor tyrosine inhibitors useful in the treatment of diseases mediated by class 3 and class 5 receptor tyrosine kinases. The compounds of this invention have also been found to be inhibitors of Pim-1.

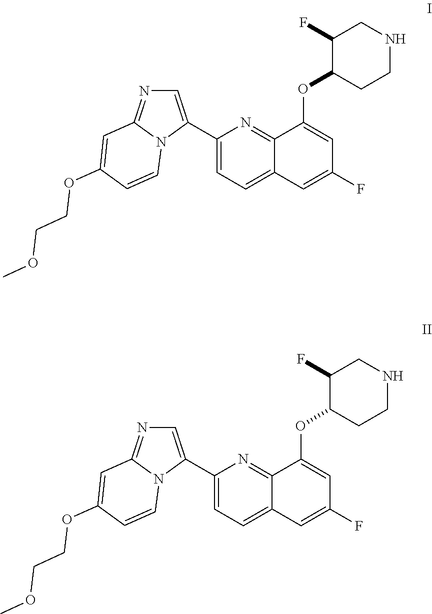

The present invention relates to novel compounds, to pharmaceutical compositions comprising the compounds, to a process for making the compounds and to the use of the compounds in therapy. More particularly, it relates to certain imidazopyridine compounds useful in the treatment and prevention of diseases mediated by class 3 and class 5 receptor tyrosine kinases. Particular compounds of this invention have also been found to be inhibitors of Pim-1.

Receptor tyrosine kinases (RTK's) include the class 3 receptor tyrosine kinases (PDGF-α, PDGFR-β, MCSF-1R, c-kit, and FLT3) and the class 5 receptor tyrosine kinases (VEGFR and KDR). It is known that such kinases are frequently aberrantly expressed in common human cancers, such as breast cancer, gastrointestinal cancer such as colon, rectal or stomach cancer, leukemia, and ovarian, bronchial or pancreatic cancer, renal cell carcinoma and gliomas.

FLT3 (fms-like tyrosine kinase; also known as Flk-2) is a member of the class 3 receptor tyrosine kinase (RTK) family, and is presumed to be involved in the hematopoietic system (Rosnet, et al., 1991, Genomics 9:380-385, Rosnet, et al., 1993, Blood 82:1110-1119). Aberrant expression of the FLT3 gene has been documented in both adult and childhood leukemias including acute myeloid leukemia (AML), AML with trilineage myelodysplasia (AML/TMDS), acute lymphoblastic leukemia (ALL), and myelodysplastic syndrome (MDS). Activating mutations of the FLT3 receptor have been found in about 35% of patients with acute myeloblastic leukemia (AML), and are associated with a poor prognosis. These types of mutations are associated with constitutive activation of the tyrosine kinase activity of FLT3, and result in proliferation and viability signals in the absence of ligand. Patients expressing the mutant form of the receptor have been shown to have a decreased chance for cure. In addition to activating mutations, ligand dependent (autocrine or paracrine) stimulation of over-expressed wild-type FLT3 contributes to AML. Thus, there is accumulating evidence for a role for hyper-activated (mutated) FLT3 kinase activity in human leukemias and myelodysplastic syndrome. FLT3 inhibitors may also be useful for treating immune related disorders and is involved in the process of angiogenesis through its expression in pericytes.

PDGFR is expressed on early stem cells, mast cells, myeloid cells, mesenchymal cells, and smooth muscles cells. PDGFR-β has been implicated in myeloid leukemias. Recently, it was shown that activating mutations in PDGFR-α kinase domain are in gastrointestinal stromal tumors (GIST) (Wong et al., 2007, Histopathology 51(6): 758-762).

In addition, blockade of PDGF signaling has been shown to reduce the development of fibrosis in various experimental models (Yoshiji et al., 2006, International Journal Molecular Medicine 17: 899-904).

Accordingly, it has been recognized that inhibitors of receptor tyrosine kinases are useful as inhibitors of the growth of mammalian cancer cells or for treating immune related disorders.

The Pim kinases are a family of three distinct vertebrate protein serine/threonine kinases (Pim-1, -2 and -3) belonging to the calmodulin-dependent protein kinase-related (CAMK) group. The over-expression of Pim-1 has been reported in various human lymphomas and acute leukemias (Amson, R. et al, Proc. Natl. Acad. Sci. U.S.A., 1989, 86: 8857-8861). In addition, there is evidence that Pim-1 is over-expressed in prostatic neoplasia and human prostate cancer (Valdman, A. et al, The Prostate, 2004, 60: 367-371; Cibull, T. L. et al, J. Clin. Pathol., 2006, 59: 285-288) and may serve as a useful biomarker in identification of prostate cancer (Dhanasekaran, S. M. et al, Nature, 2001, 412(13): 822-826). Recently, it has been discovered that Pim-1 is up-regulated by Flt-3 and may play an ancillary role in Flt-3 mediated cell survival (Kim, K. T. et al Neoplasia, 2005, 105(4): 1759-1767). Since Flt-3 itself is implicated in leukemias like AML, additional knockdown of Pim-1 may be a useful approach to treating leukemias driven by Flt-3 or various mutations. Accordingly, Pim-1 inhibitors may be useful as therapeutic agents for a variety of cancers such as hematological cancers.

Tyrosine kinase inhibitors are known in the art. U.S. Pat. No. 7,125,888 describes certain imidazo[1,2-a]pyridine compounds substituted at the 3 position with a pyridyl, thiazolyl, oxazolyl or phenyl group and at the 7 position with an optionally substituted phenyl or pyridone group, which are purported to be tyrosine kinase inhibitors. U.S. patent publication 2005/0124637 discloses certain purine derivatives as inhibitors of receptor tyrosine kinases, including FLT3. PCT publication number WO 01/40217 and U.S. Pat. No. 7,019,147 disclose certain benzimidazole compounds having activity as tyrosine kinase inhibitors.

It has now been found that a certain imidazo[1,2-a]pyridine compound bearing a quinolinyl group at the 3 position of the imidazopyridine ring is an inhibitor of receptor tyrosine kinases, in particular class 3 and class 5 receptor tyrosine kinases, which are useful for treating diseases mediated by class 3 and class 5 receptor tyrosine kinases, such as cancers, fibrosis, sclerosis, autoimmune disorders and scleroderma.

In one aspect, the invention provides a compound having the Formula I:

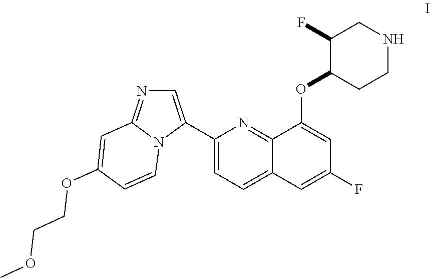

or a pharmaceutically acceptable salt thereof. The compound may also be described by the chemical name cis-6-fluoro-8-(3-fluoropiperidin-4-yloxy)-2-(7-(2-methoxyethoxy)imidazo[1,2-a]pyridin-3-yl)quinoline.

It will be appreciated that the compounds of Formula I contain one or more centers of asymmetry and may therefore be prepared and isolated in a mixture of isomers such as a racemic mixture, or in an enantiomerically pure form.

Accordingly, in one embodiment of the invention, the compound of Formula I can be represented by the Formula IA

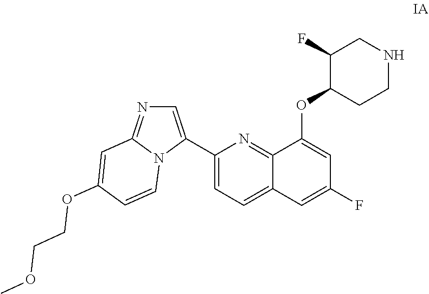

or a pharmaceutically acceptable salt thereof. The compound of Formula IA may also be described by the chemical name 6-fluoro-8-((3S,4R)-3-fluoropiperidin-4-yloxy)-2-(7-(2-methoxyethoxy)imidazo[1,2-a]pyridin-3-yl)quinoline.

In another embodiment, the compound of Formula I can be represented by the Formula IB:

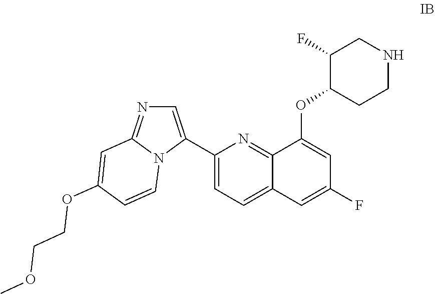

or a pharmaceutically acceptable salt thereof. The compound of Formula IB may also be described by the chemical name 6-fluoro-8-((3R,4S)-3-fluoropiperidin-4-yloxy)-2-(7-(2-methoxyethoxy)imidazo[1,2-a]pyridin-3-yl)quinoline.

The invention further relates to a compound of Formula II

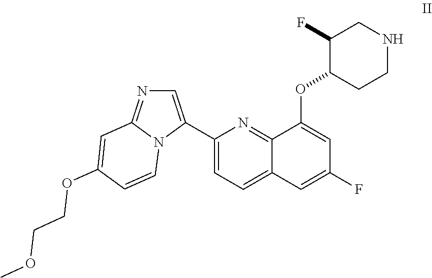

or a pharmaceutically acceptable salt thereof. The compound may also be described by the chemical name 6-fluoro-8-(trans-3-fluoropiperidin-4-yloxy)-2-(7-(2-methoxyethoxy)imidazo[1,2-a]pyridin-3-yl)quinoline.

It will be appreciated that the compounds of Formula II contain one or more centers of asymmetry and may therefore be prepared and isolated in a mixture of isomers such as a racemic mixture, or in an enantiomerically pure form.

Accordingly, in one embodiment of the invention, the compound of Formula I can be represented by the Formula IIA

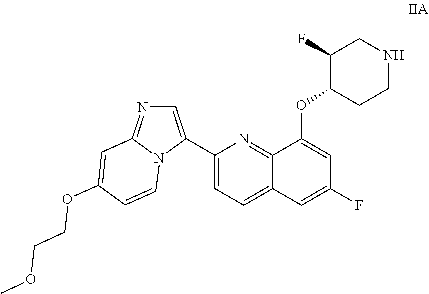

or a pharmaceutically acceptable salt thereof. The compound of Formula IIA may also be described by the chemical name 6-fluoro-8-((3S,4S)-3-fluoropiperidin-4-yloxy)-2-(7-(2-methoxyethoxy)imidazo[1,2-a]pyridin-3-yl)quinoline.

In another embodiment, the compound of Formula II can be represented by the Formula IIB:

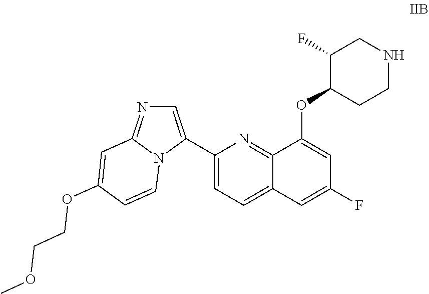

or a pharmaceutically acceptable salt thereof. The compound of Formula IIB may also be described by the chemical name 6-fluoro-8-((3R,4R)-3-fluoropiperidin-4-yloxy)-2-(7-(2-methoxyethoxy)imidazo[1,2-a]pyridin-3-yl)quinoline.

The compounds of Formulas I and II have been found to be class 3 receptor tyrosine kinase inhibitors and are useful in the treatment of cancers, such as hematological cancers (e.g., leukemias such as AML), breast cancer, colon cancer, gliomas, fibrosis (including liver fibrosis and lung fibrosis), and scleroderma.

The compounds of Formulas I and II have also been found to be inhibitors of FLT3.

It will further be appreciated that the compounds of Formulas I and II or their salts may be isolated in the form of solvates, and accordingly that any such solvate is included within the scope of the present invention.

The compounds of Formulas I and II include pharmaceutically acceptable salts thereof. In addition, the compounds of Formula I also include other salts of such compounds which are not necessarily pharmaceutically acceptable salts, and which may be useful as intermediates for preparing and/or purifying compounds of Formulas I and II and/or for separating enantiomers of compounds of Formulas I and II.

According to another aspect, the present invention provides a process for the preparation a compound of Formula I or a salt thereof as defined herein which comprises:

coupling a compound having formula III

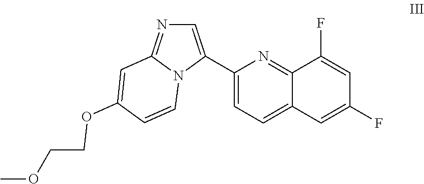

with a compound having formula IV

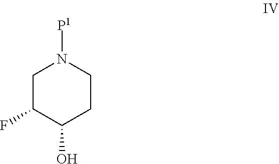

where P¹ is an amine protecting group, in the presence of a catalyst or a base, followed by removing the protecting group and forming a salt, if desired.

Suitable bases include alkali metal alkoxides, such as sodium t-butoxide.

A compound of Formula III can be prepared by treating a compound having formula V

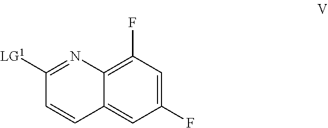

where LG¹ is a leaving group or atom, with a compound having formula VI

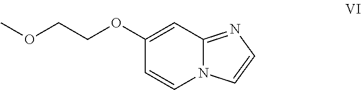

in the presence of a palladium catalyst. Examples of leaving groups include triflate. Examples of leaving atoms include Cl. The reaction is conveniently performed in the presence of a base. Suitable solvents include dioxane. Suitable catalysts include PdCl₂(PPh₃)₂ or Pd(OAc)₂ or mixtures thereof. Suitable bases include alkali metal carbonates, such as cesium or potassium carbonate.

Compound of formula V can be prepared by treating a compound having formula VII

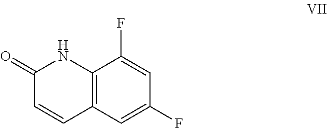

with a reagent that will incorporated a leaving group or atom. For example, when the leaving atom is Cl, the compound VII is reacted with oxalyl chloride. When the leaving group is triflate, the compound VII is reacted with triflic anhydride, preferably in the presence of a base.

The invention further provides a method of preparing a compound of Formula II, comprising:

coupling a compound having the formula VIII

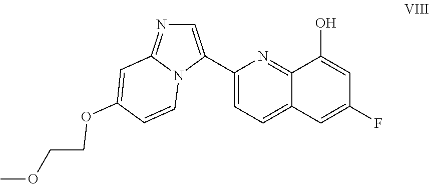

with a compound having the formula IX

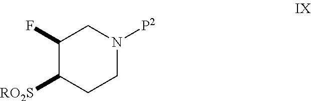

where P² is an amine protecting group and RSO₂ is an alkyl or aryl sulfonate, in the presence of base, followed by removing the protecting group and forming a salt, if desired. Examples of the R group include methyl, phenyl, or m-nitrophenyl. Suitable bases include alkali metal carbonates such as cesium or potassium carbonate. The reaction is conveniently performed at elevated temperatures, for example from 75-100° C. Suitable solvents include DMF or water.

Compounds of formula VIII can be prepared by coupling a compound having the formula X

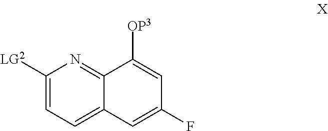

where P³ is a hydroxy protecting group and LG² is a leaving group or atom, with a compound having the formula VI

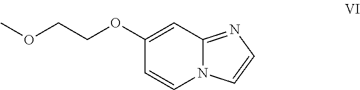

in the presence of a palladium catalyst. Examples of leaving groups include triflate. Examples of leaving atoms include Cl. The reaction is conveniently preformed in the presence of a base. Suitable solvents include dioxane. Suitable catalysts include PdCl₂(PPh₃)₂ or Pd(OAc)₂ or mixtures thereof. Suitable bases include alkali metal carbonates, such as cesium or potassium carbonate.

A compound of the formula X can be prepared by treating a compound having the formula XI

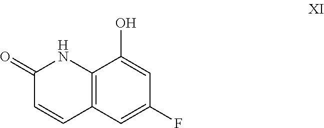

with a hydroxy protecting group, followed by treatment with a reagent that will incorporated a leaving group or atom. For example, when the leaving atom is Cl, the protected compound XI is reacted with oxalyl chloride. When the leaving group is triflate, the protected compound XI is reacted with triflic anhydride, preferably in the presence of a base. Suitable solvents include DMF and DME. The reaction is conveniently preformed at elevated temperatures.

The compounds of Formulas I and II are useful for treating diseases and disorders mediated by class 3 and/or class 5 receptor tyrosine kinases. In particular embodiments, compounds of this invention are inhibitors of one or more of the class 3 receptor tyrosine kinases, for example PDGFR and FLT3. For example, compounds of this invention are useful in the treatment fibrosis (including lung, liver and kidney fibroses), scleroderma, and cancers, including hematological malignancies.

As used herein, the term treatment includes prophylaxis as well as treatment of an existing condition.

Examples of hematological malignancies include, for instance, leukemias, lymphomas (non-Hodgkin's lymphoma), Hodgkin's disease (also called Hodgkin's lymphoma), and myeloma—for instance, acute lymphocytic leukemia (ALL), acute myeloid leukemia (AML), acute promyelocytic leukemia (APL), chronic lymphocytic leukemia (CLL), chronic myeloid leukemia (CML), chronic neutrophilic leukemia (CNL), acute undifferentiated leukemia (AUL), anaplastic large-cell lymphoma (ALCL), prolymphocytic leukemia (PML), juvenile myelomonocyctic leukemia (JMML), adult T-cell ALL, AML with trilineage myelodysplasia (AML/TMDS), mixed lineage leukemia (MLL), myelodysplastic syndromes (MDSs), myeloproliferative disorders (MPD), and multiple myeloma (MM).

Particular examples of PDGFR-driven or dependent cancers which may be treated with compounds of this invention include dermatofibrosarcoma protuberans (DFSB), chronic myelomonocytic leukemia (CMML), hypereosinophilic syndrome (HES), glioblastoma multiforme (GBM) and gastrointestinal stromal tumors (GIST).

FLT3 inhibitors may also be useful for treating immune related disorders such as bone marrow transplant rejection, solid organ rejection after transplant, ankylosing spondylitis, arthritis, aplastic anemia, Behcet's disease, Graves' disease, hemolytic anemia, hyper IgE syndrome, idiopathic thrombocytopenia purpura (ITP), multiple sclerosis (MS), rheumatoid arthritis, Wegener's granulomatosis, type 1 diabetes mellitus, Myasthenia gravis, and psoriasis.

Particular compounds of this invention are inhibitors of Pim-1 and therefore are useful in treating diseases and disorders mediated by Pim-1, such as cancers such as hematological cancers.

Accordingly, another aspect of this invention provides a method of treating diseases or medical conditions in a mammal mediated by a class 3 and/or class 5 receptor tyrosine kinase, comprising administering to said mammal one or more compounds of Formula I and/or II or a pharmaceutically acceptable salt or prodrug thereof in an amount effective to treat or prevent said disorder.

Another aspect of this invention provides a method of treating diseases or medical conditions in a mammal mediated by Pim-1, comprising administering to said mammal one or more compounds of Formula I and/or II or a pharmaceutically acceptable salt or prodrug thereof in an amount effective to treat or prevent said disorder.

The phrase “effective amount” means an amount of compound that, when administered to a mammal in need of such treatment, is sufficient to (i) treat or prevent a particular disease, condition, or disorder mediated by a class 3 receptor tyrosine kinase, (ii) attenuate, ameliorate, or eliminate one or more symptoms of the particular disease, condition, or disorder, or (iii) prevent or delay the onset of one or more symptoms of the particular disease, condition, or disorder described herein.

The amount of a compound of Formula I or II that will correspond to such an amount will vary depending upon factors such as the particular compound, disease condition and its severity, the identity (e.g., weight) of the mammal in need of treatment, but can nevertheless be routinely determined by one skilled in the art.

As used herein, the term “mammal” refers to a warm-blooded animal that has or is at risk of developing a disease described herein and includes, but is not limited to, guinea pigs, dogs, cats, rats, mice, hamsters, and primates, including humans.

Compounds of the present invention can be used in combination with one or more additional drugs, for example an anti-inflammatory compound, anti-fibrotic compound or a chemotherapeutic that works by the same or by a different mechanism of action.

Compounds of the invention may be administered by any convenient route, e.g. into the gastrointestinal tract (e.g. rectally or orally), the nose, lungs, musculature or vasculature, or transdermally or dermally. The compounds may be administered in any convenient administrative form, e.g. tablets, powders, capsules, solutions, dispersions, suspensions, syrups, sprays, suppositories, gels, emulsions, patches etc. Such compositions may contain components conventional in pharmaceutical preparations, e.g. diluents, carriers, pH modifiers, sweeteners, bulking agents, and further active agents. If parenteral administration is desired, the compositions will be sterile and in a solution or suspension form suitable for injection or infusion. Such compositions form a further aspect of the invention.

According to another aspect, the present invention provides a pharmaceutical composition, which comprises a compound of Formula I or a pharmaceutically acceptable salt thereof, as defined hereinabove. In one embodiment, the pharmaceutical composition includes the compound of Formula I together with a pharmaceutically acceptable diluent or carrier.

According to another aspect, the present invention provides a pharmaceutical composition, which comprises a compound of Formula II or a pharmaceutically acceptable salt thereof, as defined hereinabove. In one embodiment, the pharmaceutical composition includes the compound of Formula II together with a pharmaceutically acceptable diluent or carrier.

According to another aspect, the present invention provides a compound of Formula I or a pharmaceutically acceptable salt thereof, for use in therapy, such as the treatment of a class 3 receptor tyrosine kinase-mediated condition.

According to another aspect, the present invention provides a compound of Formula II or a pharmaceutically acceptable salt thereof, for use in therapy, such as the treatment of a class 3 receptor tyrosine kinase-mediated condition.

In certain embodiments, the invention provides a compound of Formula I or a pharmaceutically acceptable salt thereof, for use in the treatment of cancer.

In certain embodiments, the invention provides a compound of Formula II or a pharmaceutically acceptable salt thereof, for use in the treatment of cancer.

In certain embodiments, the invention provides a compound of Formula I or a pharmaceutically acceptable salt thereof, for use in the treatment of fibrosis.

In certain embodiments, the invention provides a compound of Formula II or a pharmaceutically acceptable salt thereof, for use in the treatment of fibrosis.

In certain embodiments, the invention provides a compound of Formula I or a pharmaceutically acceptable salt thereof, for use in the treatment of scleroderma.

In certain embodiments, the invention provides a compound of Formula II or a pharmaceutically acceptable salt thereof, for use in the treatment of scleroderma.

According to another aspect, the present invention provides a compound of Formula I or a pharmaceutically acceptable salt thereof, for use in therapy, such as the treatment of a Pim-1-mediated condition.

According to another aspect, the present invention provides a compound of Formula II or a pharmaceutically acceptable salt thereof, for use in therapy, such as the treatment of a Pim-1-mediated condition.

According to a further aspect, the present invention provides the use of a compound of Formula I or a pharmaceutically acceptable salt thereof, in the manufacture of a medicament to treat a class 3 receptor tyrosine kinase-mediated condition.

According to a further aspect, the present invention provides the use of a compound of Formula II or a pharmaceutically acceptable salt thereof, in the manufacture of a medicament to treat a class 3 receptor tyrosine kinase-mediated condition.

According to a further aspect, the present invention provides the use of a compound of Formula I or a pharmaceutically acceptable salt thereof, in the manufacture of a medicament to treat a Pim-1-mediated condition.

According to a further aspect, the present invention provides the use of a compound of Formula II or a pharmaceutically acceptable salt thereof, in the manufacture of a medicament to treat a Pim-1-mediated condition.

### EXAMPLES

The following examples illustrate the invention. In the examples described below, unless otherwise indicated all temperatures are set forth in degrees Celsius. Reagents were purchased from commercial suppliers such as Aldrich Chemical Company, Lancaster, TCI or Maybridge, and were used without further purification unless otherwise indicated. Tetrahydrofuran (THF), dichloromethane (DCM, methylene chloride), toluene, and dioxane were purchased from Aldrich in Sure seal bottles and used as received.

The reactions set forth below were done generally under a positive pressure of nitrogen or argon or with a drying tube (unless otherwise stated) in anhydrous solvents, and the reaction flasks were typically fitted with rubber septa for the introduction of substrates and reagents via syringe. Glassware was oven dried and/or heat dried.

### Example A

### Cellular PDGFR Assay

The ability of compounds of this invention to inhibit PDGF-induced PDGFR phosphorylation was assessed by using mouse NIH3T3 cells.

25,000 cells in DMEM supplemented with 10% fetal bovine serum were added to each well of a black 96-well cell culture plate. Plates were incubated in a 37° C./5% CO₂ incubator for 6-8 hours. Plates were then washed and incubated with serum-free DMEM, and the cells were returned to the 37° C./5% CO₂ incubator for 16-20 hours.

Compound test solutions were added at a final concentration of 0.5% DMSO, and the cells were incubated in a 37° C./5% CO₂ incubator for 1 hour. PDGF-BB ligand was then added (75 ng/mL) and incubated for 15 minutes. Cells were washed with PBS and fixed in 3.7% formaldehyde in PBS for 10 minutes. This was followed by washing in PBS/0.2% Triton X-100 and permeabilizing in 100% MeOH for 10 minutes. Cells were blocked in Odyssey blocking buffer (LI-COR Biosciences) for 1 hour. Antibodies to phosphorylated PDGFRβ and total PDGFRβ were added to the cells and incubated for 3 hours. After washing with PBS/0.2% TritonX-100, the cells were incubated with fluorescently-labeled secondary antibodies (goat anti-rabbit IgG-IRDye800 and goat anti-mouse IgG-Alexa Fluor 680) for an additional hour. Cells were then washed with PBS and analyzed for fluorescence at both wavelengths using the Odyssey Infrared Imaging System (LI-COR Biosciences). Phosphorylated PDGFR signal was normalized to total PDGFR signal. Compounds of this invention had IC₅₀'s values less than 10 μM in this assay.

### Example B

### Cellular FLT3 Assay

The inhibition of FLT3 ligand (FL)-induced phosphorylated FLT3 in human RS4; 11 cells was measured as follows. Cells were plated in 96-well V-bottom plates in RPMI/10% FCS at a concentration of 1 million cells/well. Diluted compounds were added at a final concentration of 0.5% DMSO for one hour. FL was added at a final concentration of 50 ng/ml. After a 15 minute incubation, the cells were pelleted by centrifugation and resuspended in lysis buffer. Phospho-FLT3 was detected by standard ELISA procedure (R&D Systems; DYC368). Briefly, after 20 minutes on ice, the lysate was added to 96-well plates coated with capture antibody to total FLT3. Phospho-FLT3 was detected by the addition of antibody to phospho-tyrosine conjugated to HRP. After addition of substrate and stop solution, the signal was read at A450. Compounds of this invention had IC₅₀'s values less than 10 μM in this assay.

### Example 1

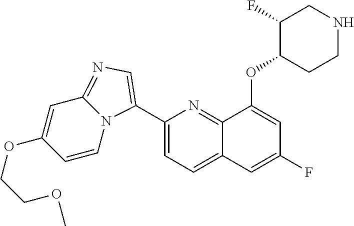

### Non-racemic 6-fluoro-8-(cis-3-fluoropiperidin-4-yloxy)-2-(7-(2-methoxyethoxy)imidazo[1,2-a]pyridin-3-yl)quinoline, Enantiomer 2

Step 1A: Preparation of 2-chloro-4-(2-methoxyethoxy)pyridine: A mixture of 2-chloro-4-nitropyridine (43.6 g, 275.0 mmol) and 2-methoxyethanol (325.6 ml, 425 mmol) was cooled to 0° C. Potassium 2-methylpropan-2-olate (35.73 g, 302.5 mmol) was added and the resulting mixture was stirred while warming to ambient temp over 2 hours. The reaction mixture was concentrated under reduced pressure followed by dilution with 500 ml of water. The resulting mixture was extracted twice with 250 ml of dichloromethane. The combined organic layers were dried over MgSO₄ and concentrated under reduced pressure to produce the desired compound as a golden oil. (50.2 g, 97% yield) MS APCI (+) m/z 188 and 189.9 (M+1 of each isotope) detected.

Step 1B: Preparation of 4-(2-methoxyethoxy)pyridin-2-amine: A steady stream of nitrogen was passed through a mixture of 2-chloro-4-(2-methoxyethoxy)pyridine (50.17 g, 267.4 mmol), Pd₂ dba₃ (4.897 g, 5.348 mmol), XPHOS (5.099 g, 10.70 mmol) and tetrahydrofuran (445.7 ml) for 10 minutes. To the resulting degassed mixture was added lithium bis(trimethylsilyl)amide (561.5 ml, 561.5 mmol). After addition, the resulting mixture was heated to 60° C. for 18 hours. The reaction was cooled to ambient temperature and diluted with 1 N hydrochloric acid (200 mL). The resulting solution was washed twice with 500 ml of methyl-tert-butyl ether. The pH of the aqueous layer was taken to 11 with 6 N NaOH and was extracted with dichloromethane (3×500 ml). The combined organic layers were dried over MgSO₄ and concentrated under reduced pressure to yield title compound. (35 g, 78% yield) MS APCI (+) m/z 169 (M+1) detected.

Step 1C: Preparation of 7-(2-methoxyethoxy)imidazo[1,2-a]pyridine: A mixture of 4-(2-methoxyethoxy)pyridin-2-amine (20.0 g, 119 mmol), 2-chloroacetaldehyde (32.2 ml, 250 mmol) and tetrahydrofuran (100 mL) were heated in a sealed tube to 75° C. over 3 days. The reaction mixture was concentrated under reduced pressure and dissolved in ethyl acetate. The resulting solution was washed twice with saturated sodium bicarbonate. The combined organic layers were dried over MgSO₄ and concentrated under reduced pressure to yield title compound. (23.5 g, quantitative yield) MS APCI (+) m/z 193 (M+1) detected.

Step 2A: Preparation of N-(2,4-difluorophenyl)cinnamamide: Cinnamoyl chloride (13 g, 77 mmol) was added dropwise to a solution of pyridine (5.6 ml, 70 mmol) and (2,4-difluoroaniline (9.0 g, 70 mmol) in CH₂Cl₂ (0.5 M, 140 mL). After 12 hours, saturated NaHCO₃ was added, the organic phase was washed with 1 N HCl, dried over Na₂SO₄, filtered and condensed to afford white solids with pinkish tint. About 150 mL or DCM were added and the mixture was stirred 30 minutes. About 150 mL of hexanes were added, and the mixture was filtered to collect white solids (13.5 g).

Step 2B: Preparation of 6,8-difluoroquinolin-2(1H)-one: N-(2,4-difluorophenyl)cinnamamide (1.49 g, 5.75 mmol) was mixed with aluminum trichloride (2.30 g, 17.2 mmol) then heated to 210° C. for 40 minutes. The mixture was cooled to ambient temperature, and ice water was added. The resultant solids were collected by filtration, washed with water and dried under high vacuum for 12 hours to obtain 6,8-difluoroquinolin-2(1H)-one (1.04 g).

Step 2C: Preparation of 2-chloro-6,8-difluoroquinoline: 2 M Oxalyl dichloride in dichloromethane (3.59 ml, 7.19 mmol) was added dropwise to a mixture of 6,8-difluoroquinolin-2(1H)-one (0.434 g, 2.40 mmol) in 1,2-dichloroethane (24 ml). 1 drop of DMF, (gas evolution) was added, and the mixture was heated to 70° C. for 30 minutes. The mixture was cooled to ambient temperature, and saturated NaHCO₃ was added. The mixture was extracted twice with CH₂Cl₂, the combined organic phases were dried over Na₂SO₄, filtered and condensed. The residue was purified by flash column chromatography to provide 2-chloro-6,8-difluoroquinoline (0.388 g, 1.94 mmol).

Step 3A: Preparation of benzyl 4-(trimethylsilyloxy)-5,6-dihydropyridine-1(2H)-carboxylate: To benzyl 4-oxopiperidine-1-carboxylate (152 g, 650 mmol) in DMF (650 mL) was added TMS-Cl (148 ml, 117 mmol) followed by triethylamine (326 ml, 234 mmol). The slurry was warmed to 80° C. for 16 hours, diluted with hexanes (about 1 L), washed 3 times with saturated NaHCO₃ solution, dried, filtered and concentrated to obtain benzyl 4-(trimethylsilyloxy)-5,6-dihydropyridine-1(2H)-carboxylate (199 g, 652 mmol) as a orange oil.

Step 3B: Preparation of benzyl 3-fluoro-4-oxopiperidine-1-carboxylate: Selectfluor® (181.2 g, 511.4 mmol) was added portion-wise (about 25 g portions) to a ice cold solution of benzyl 4-(trimethylsilyloxy)-5,6-dihydropyridine-1(2H)-carboxylate (142 g, 465 mmol) in CH₃CN (2 L) over approx 30 minutes. The ice bath was removed and the mixture was allowed to stand for 12 hours. The mixture was concentrated to a slurry, diluted with EtOAc and brine and the layers were separated. The brine phase was extracted once with EtOAc, and the combined organic phases were washed with saturated NaHCO₃ and brine, dried (Na₂SO₄), filtered and condense to isolate 112 g of a dark thick oil.

Step 3C: Preparation of cis-benzyl 3-fluoro-4-hydroxypiperidine-1-carboxylate: L-selectride® (663 ml, 663 mmol) was added drop-wise to an ice cold solution of benzyl 3-fluoro-4-oxopiperidine-1-carboxylate (138.9 g, 552.8 mmol) in anhydrous THF (421 mL). The ice bath was removed and the reaction was allowed to stand for 12 hours. The reaction mixture was carefully added (drop-wise, via addition funnel) to a vigorously stirring mixture of 80 mL MeOH, 2 N NaOH (1400 mL) H₂O₂ (376 mL, 50%) in a large amount of ice, taking care to control the exotherm. The mixture was stirred for 12 hours, then about 2 L of EtOAc were added. The mixture was stirred an additional 1 hour. The layers were separated and the aqueous phase was extracted with EtOAc. The combined organic phases were dried over Na₂SO₄, filtered and condense to afford 112 g of oil. The residue was purified by flash column chromatography (silica gel, eluting with a gradient of 30% EtOAc to 75% EtOAc in hexanes) to afford 38 g of the title compound.

Step 3D: Preparation of non-racemic cis-benzyl 3-fluoro-4-hydroxypiperidine-1-carboxylate: A 32 g sample of the material of Step 3C was separated by chiral SFC separation (3 cm×15 cm Chiralpak AD-H column; mobile phase 22% Ethanol/CO₂, 100 bar; flow rate 100 mL/min; 50 mg/mL injections, 1.5 mL injection volume; 220 nM) to afford first eluting peak (Peak 1; 11.2 g, Rt 2.63 min) in >99% ee and second eluting peak (Peak 2; 11.8 g, Rt 4.01 min) in >99% ee.

Step 4A: Preparation of 6,8-difluoro-2-(7-(2-methoxyethoxy)imidazo[1,2-a]pyridin-3-yl)quinoline: A flask was charged with 7-(2-methoxyethoxy)imidazo[1,2-a]pyridine (0.143 g, 0.742 mmol), 2-chloro-6,8-difluoroquinoline (0.148 g, 0.742 mmol), K₂CO₃ (0.205 g, 1.48 mmol), Pd(OAc)₂ (0.008 g, 0.037 mmol), Pd(PPh₃)₄ (0.043 g, 0.037 mmol), degassed dioxane (5 mL) and water (0.5 mL), and the mixture was heated to 100 C for 12 h. The reaction was cooled to ambient temperature, diluted with CH₂Cl₂, filtered through a pad of Celite®, and condensed. The residue was purified by flash column chromatography to obtain 6,8-difluoro-2-(7-(2-methoxyethoxy)imidazo[1,2-a]pyridin-3-yl)quinoline (0.204 g, 0.574 mmol). MS APCI (+) m/z 356.2 (M+1) detected.

Step 4B: Preparation of non-racemic benzyl cis-3-fluoro-4-(6-fluoro-2-(7-(2-methoxyethoxy)imidazo[1,2-a]pyridin-3-yl)quinolin-8-yloxy)piperidine-1-carboxylate, Enantiomer 2: Non-racemic benzyl cis-3-fluoro-4-hydroxypiperidine-1-carboxylate (0.089 g, 0.35 mmol, peak 2 isolated in step 3D) was added to DMF (0.45 mL) and the mixture was cooled to 0° C. 1 M Potassium t-butoxide in THF was added dropwise (0.34 ml, 0.34 mmol), and the reaction was warmed to ambient temperature and stirred for 15 minutes. 6,8-Difluoro-2-(7-(2-methoxyethoxy)imidazo[1,2-a]pyridin-3-yl)quinoline (0.062 g, 0.18 mmol) in DMF (0.45 mL) was added and the reaction was allowed to stand at ambient temperature for 120 hours. The entire reaction mixture was applied directly to a silica gel samplet and the mixture was purified by flash chromatography on SiO₂ by eluting with a 1 to 20% gradient using (6% NH₄OH in MeOH)/ethyl acetate, to obtain the title compound. MS APCI (+) m/z 589.3.

Step 4C: Preparation of non-racemic 6-fluoro-8-(cis-3-fluoropiperidin-4-yloxy)-2-(7-(2-methoxyethoxy)imidazo[1,2-a]pyridin-3-yl)quinoline, Enantiomer 2: A flask was charged with non-racemic benzyl cis-3-fluoro-4-(6-fluoro-2-(7-(2-methoxyethoxy)imidazo[1,2-a]pyridin-3-yl)quinolin-8-yloxy)piperidine-1-carboxylate, Enantiomer 2 (0.050 g, 0.086 mmol), ammonium formate (0.11 g, 1.7 mmol) and 10% Pd/C (0.009 g, 0.009 mmol) [Aldrich, wet, Degussa type E101 NEW] and the mixture was slurried in EtOH (95%, 2.5 mL) and heated to reflux for 40 minutes. The mixture was cooled to ambient temperature, filtered through a nylon membrane filtered (0.45 μM) using additional EtOH and then concentrate in vacuo. The residue was dissolved in water and ethyl acetate. Solid NaHCO₃ (50 mg) was added to adjust the aqueous layer to approximately pH 8. The layers were separated and the aqueous layer was extracted with EtOAc. The organic layers were combined and washed with saturated NaCl. The organic layer was dried over Na₂SO₄ and concentrate in vacuo. The residue was purified by silica gel chromatography, eluting with a 1-25% gradient of (2% NH₄OH in isopropanol)/methylene chloride to provide the desired material as a light yellow solid (11.9 mg). The specific rotation of this sample is −62.1 degrees in chloroform at 20° C. MS APCI (+) m/z 455.3.

### Example 2

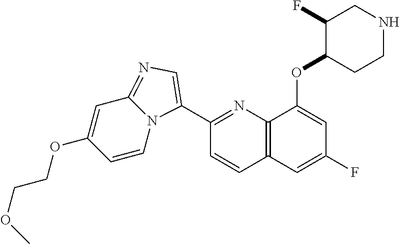

Non-racemic 6-fluoro-8-(cis-3-fluoropiperidin-4-yloxy)-2-(7-(2-methoxyethoxy)imidazo[1,2-a]pyridin-3-yl)quinoline, Enantiomer 1

Prepared according to the procedure in Example 1, using non-racemic benzyl cis-3-fluoro-4-hydroxypiperidine-1-carboxylate, Enantiomer 1, which was prepared from non-racemic cis-benzyl 3-fluoro-4-hydroxypiperidine-1-carboxylate, peak 1 (which was isolated in Example 1, step 3D), in place of non-racemic benzyl cis-3-fluoro-4-hydroxypiperidine-1-carboxylate, Enantiomer 2. MS APCI (+) m/z 455.2. The specific rotation of this sample is +63.2 degrees in chloroform at 20° C.

### Example 3

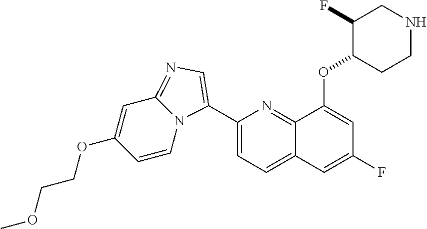

### Non-racemic 6-fluoro-8-(trans-3-fluoropiperidin-4-yloxy)-2-(7-(2-methoxyethoxy)imidazo[1,2-a]pyridin-3-yl)quinoline

Step 1A: Preparation of N-(4-fluoro-2-hydroxyphenyl)cinnamamide: 2-Amino-5-fluorophenol (33.1 g, 260.4 mmol) dissolved in dichloromethane (500 mL) and pyridine (42.12 ml, 520.8 mmol) and cooled to 0° C. was treated dropwise with cinnamoyl chloride (43.38 g, 260.4 mmol) dissolved in dichloromethane (250 mL) over 1 hour. The reaction mixture was warmed to ambient temperature and stirred for 16 hours. Water (2 mL) was added and the reaction was stirred for 1 hour, then diluted with 5% HCl (about. 250 mL) and concentrated. The resulting aqueous layer was diluted with methylene chloride (750 mL) and stirred vigorously. The solids were filtered, washed with methylene chloride, water and methylene chloride, then air-dried to provide a dark green solid, (46.47 g). ¹H NMR (400 MHz, CDCl₃ with CD₃OD) δ 7.76 (d, J=15.3 Hz, 1H), 7.60-7.52 (m, 2H), 7.44-7.33 (m, 4H), 6.74-6.68 (m, 1H), 6.66 (d, J=15.6 Hz, 1H), 6.62-6.56 (m, 1H).

Step 1B: Preparation of 6-fluoroquinoline-2,8-diol: A flask was charged with N-(4-fluoro-2-hydroxyphenyl)cinnamamide (25.5 g, 99.1 mmol) and aluminum trichloride (39.7 g, 297 mmol), and the reaction was inserted into a pre-heated 210° C. bath and heated for 1.5 hours. The reaction was cooled and quenched with iced water. The resultant solids were stirred at ambient temperature for 12 hours, then collect by filtration and washed with water, 5% HCl, water, and air-dried to provide the desired product as a dark solid (18.6 g).

Step 1C: Preparation of 8-(benzyloxy)-6-fluoroquinolin-2-ol: (Bromomethyl)benzene (11.20 ml, 94.18 mmol) was added dropwise to a refluxing suspension of 6-fluoroquinoline-2,8-diol (14.06 g, 78.48 mmol) and DBU (17.61 ml, 117.7 mmol) in isopropanol (190 ml). The reaction was heated at reflux for 6 hours then cooled to ambient temperature and stirred for 60 hours. The reaction was concentrate in vacuo and the residue was suspended in CHCl₃ and wash gently with 0.4 N NaOH, 1M HCl, and water. The organic layer was dried over Na₂SO₄ and concentrated in vacuo. The residue was purified by silica gel chromatography, eluting with 3:2 hexanes/ethyl acetate then 1:1 hexanes/ethyl acetate to provide the product as a brown solid, (7.98 g). MS APCI (+) m/z 269.9.

Step 1D: Preparation of 8-(benzyloxy)-2-chloro-6-fluoroquinoline: A solution of oxalyl dichloride (2.0 M in dichloromethane) (44.5 ml, 88.9 mmol) was added to a pre-cooled (0° C.) solution of 8-(benzyloxy)-6-fluoroquinolin-2-ol (7.98 g, 29.6 mmol) slurried in 1,2-dichloroethane (125 mL) and DMF (5 drops). The reaction was warmed to ambient temperature and heated to 70° C. for 4 hours. The reaction was cooled and stirred overnight, then concentrated in vacuo. The residue was dissolved in methylene chloride, treated with 6% NaHCO₃ and separated. The aqueous was washed with methylene chloride and the combined organic layers were dried over Na₂SO₄ then concentrated in vacuo. The residue was dissolved in methylene chloride (75 mL), treated with charcoal (2.5 g) then purified by filtered chromatography on SiO₂, eluting with 4:1 hexanes/ethyl acetate then 3:2 hexanes/ethyl acetate to provide the title compound as a pale pink solid (9.6 g). ¹H NMR (400 MHz, CDCl₃) δ 9.05 (brd s, 1H), 7.71-7.64 (m, 1H), 7.49-7.36 (m, 5H), 6.91-6.82 (m, 2H), 6.75-6.68 (m, 1H), 5.17 (s, 2H).

Step 1E: Preparation of 8-(benzyloxy)-6-fluoro-2-(7-(2-methoxyethoxy)imidazo[1,2-a]pyridin-3-yl)quinoline: A flask was charged with 8-(benzyloxy)-2-chloro-6-fluoroquinoline (2.00 g, 6.95 mmol) with 7-(2-methoxyethoxy)imidazo[1,2-a]pyridine (1.34 g, 6.95 mmol), K₂CO₃ (1.92 g, 13.9 mmol), Pd(OAc)₂ (0.0780 g, 0.348 mmol) and Pd(PPh₃)₄ (0.402 g, 0.348 mmol), and dioxane (47 mL) and water (4.7 mL) were added. The mixture was degassed with argon, then heated to reflux for 33 hours and stirred at ambient temperature for 60 hours. The reaction was diluted with ethyl acetate (75 mL) and a small amount of water, then filtered through a nylon membrane. The filtrate was concentrated, and the residue was dissolved in ethyl acetate and water and the layers were separated. The aqueous layer was washed twice with ethyl acetate, and the combined organic layers were washed with saturated NaCl, dried over Na₂SO₄ and concentrate in vacuo. The residue was purified on SiO₂, eluting with a gradient of (6% NH₄OH in MeOH/ethyl acetate, to provide the desired material (1.1 g). MS APCI (+) m/z 444.2.

Step 1F: Preparation of 6-fluoro-2-(7-(2-methoxyethoxy)imidazo[1,2-a]pyridin-3-yl)quinolin-8-ol: 8-(Benzyloxy)-6-fluoro-2-(7-(2-methoxyethoxy)imidazo[1,2-a]pyridin-3-yl)quinoline (1.87 g, 4.22 mmol), ammonium formate (5.32 g, 84.3 mmol) and 10% Pd/C (0.449 g, 0.422 mmol) [wet, Degussa type E101 NEW] in EtOH (95%, 125 mL) were combined and the slurry was heated to reflux for 1 hour. The reaction was cooled to ambient temperature, diluted with CHCl₃ (400 mL) and filtered through a nylon membrane. The filtrate was concentrated in vacuo to provide the desired product as a yellow solid (1.18 g). MS APCI (+) m/z 354.2.

Step 2A: Preparation of 2-chloro-4-(2-methoxyethoxy)pyridine: A mixture of 2-chloro-4-nitropyridine (43.6 g, 275 mmol) and 2-methoxyethanol (325 ml, 425 mmol) was cooled to 0° C. Potassium 2-methylpropan-2-olate (35.7 g, 302 mmol) was added and the resulting mixture was stirred while warming to ambient temperature over 2 hours. The reaction mixture was concentrated under reduced pressure followed by dilution with 500 ml of water. The resulting mixture was extracted with dichloromethane. The combined organic layers were dried over MgSO₄ and concentrated under reduced pressure to produce the desired compound as an oil (50.2 g). MS APCI (+) m/z 188 and 189.9 (M+1 of each isotope) detected.

Step 2B: Preparation of 4-(2-methoxyethoxy)pyridin-2-amine: A steady stream of nitrogen was passed through a mixture of 2-chloro-4-(2-methoxyethoxy)pyridine (50.1 g, 267 mmol), Pd₂ dba₃ (4.89 g, 5.34 mmol), XPHOS (5.09 g, 10.7 mmol) and tetrahydrofuran (445 ml) for 10 minutes. To the resulting degassed mixture was added lithium bis(trimethylsilyl)amide (561 ml, 561 mmol). After addition, the resulting mixture was heated to 60° C. for 18 hours. The reaction was cooled to ambient temperature and diluted with 1 N hydrochloric acid (200 mL). The resulting solution was washed twice with 500 ml of methyl-tert-butyl ether. The pH of the aqueous layer was adjusted to 11 with 6 N NaOH and extracted with dichloromethane. The combined organic layers were dried over MgSO₄ and concentrated under reduced pressure to yield title compound (35 g) MS APCI (+) m/z 169 (M+1) detected.

Step 3: Preparation of non-racemic benzyl cis-3-fluoro-4-(methylsulfonyloxy)piperidine-1-carboxylate: To a solution of non-racemic (cis)-benzyl 3-fluoro-4-hydroxypiperidine-1-carboxylate (1.71 g, 6.74 mmol, prepared from non-racemic cis-benzyl 3-fluoro-4-hydroxypiperidine-1-carboxylate peak 1 which was isolated in Example 1, step 3D) in anhydrous CH₂Cl₂ (13.5 mL) and triethylamine (1.22 ml, 8.76 mmol) at 0° C. was added methanesulfonyl chloride (0.57 ml, 7.4 mmol). The reaction was warmed slowly to ambient temperature for 12 h, then partition between saturated NaHCO₃ (400 mL) and CH₂Cl₂ (400 mL). The aqueous layer was extracted with CH₂Cl₂ (2×200 mL). The combined organic phases were washed with 1 N HCl (200 mL) and brine (200 mL), dried over Na₂SO₄ and concentrate to afford a yellow powder. The residue was purified by column chromatography eluting with ethyl acetate/hexane mixture to provide the desired product which was used in Step 4A.

Step 4A: Preparation of non-racemic benzyl trans-3-fluoro-4-(6-fluoro-2-(7-(2-methoxyethoxy)imidazo[1,2-a]pyridin-3-yl)quinolin-8-yloxy)piperidine-1-carboxylate: Non-racemic benzyl cis-3-fluoro-4-(methylsulfonyloxy)piperidine-1-carboxylate (0.0784 g, 0.237 mmol), 6-fluoro-2-(7-(2-methoxyethoxy)imidazo[1,2-a]pyridin-3-yl)quinolin-8-ol (0.076 g, 0.22 mmol), Cs₂CO₃ (0.070 g, 0.22 mmol) were combined in DMF (0.4 mL) and heated to 100° C. for 24 hours. The reaction was cooled to ambient temperature, diluted with CHCl₃, filtered through celite, and condensed. The residue was purified by flash chromatography to obtain 69 mg of the title compound. MS APCI (+) m/z 589.2 (M+1) detected. This material was used in Step 4B

Step 4B: Preparation of non-racemic 6-fluoro-8-(trans-3-fluoropiperidin-4-yloxy)-2-(7-(2-methoxyethoxy)imidazo[1,2-a]pyridin-3-yl)quinoline: A flask was charged with non-racemic benzyl trans-3-fluoro-4-(6-fluoro-2-(7-(2-methoxyethoxy)imidazo[1,2-a]pyridin-3-yl)quinolin-8-yloxy)piperidine-1-carboxylate (0.068 g, 0.12 mmol), and THF (4 mL), EtOH (4 mL), 5 drops of concentrated HCl and Pearlman's Catalyst (0.049 g, 0.035 mmol) [20% wt, wet, Degussa type] were added. The reaction was placed under a balloon of H₂ and stirred overnight. The reaction was filtered through GF/F paper, condensed and purify the residue by flash chromatography to obtain the title compound (0.020 g, 0.044 mmol). MS APCI (+) m/z 455.3 (M+1) detected.

1. A compound of general Formula I: 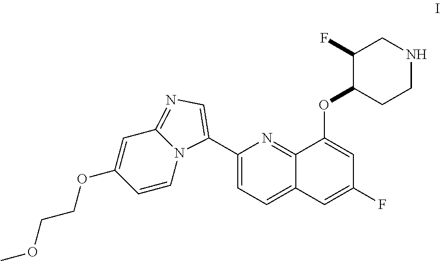
having the chemical name cis-6-fluoro-8-(3-fluoropiperidin-4-yloxy)-2-(7-(2-methoxyethoxy)imidazo[1,2-a]pyridin-3-yl)quinoline or a pharmaceutically acceptable salt thereof.

2. The compound of claim 1, having the formula IA 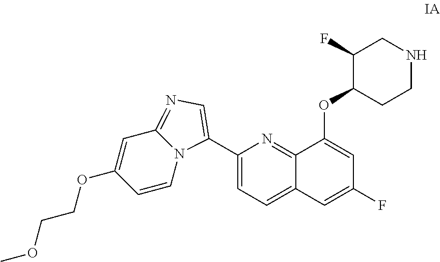
and having the chemical name 6-fluoro-8-((3S,4R)-3-fluoropiperidin-4-yloxy)-2-(7-(2-methoxyethoxy)imidazo[1,2-a]pyridin-3-yl)quinoline.

3. The compound of claim 1, having the Formula IB: 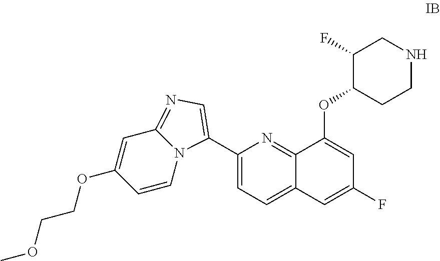
and having the chemical name 6-fluoro-843R,4S)-3-fluoropiperidin-4-yloxy)-2-(7-(2-methoxyethoxy)imidazo[1,2-a]pyridin-3-yl)quinoline.

4. A compound of Formula II 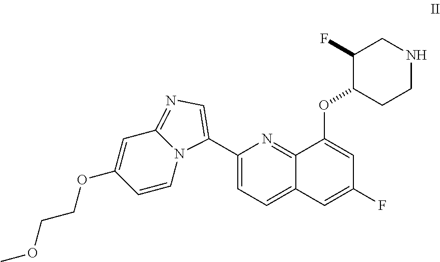
having the chemical name 6-fluoro-8-(trans-3-fluoropiperidin-4-yloxy)-2-(7-(2-methoxyethoxy)imidazo[1,2-a]pyridin-3-yl)quinoline or a pharmaceutically acceptable salt thereof.

5. The compound of claim 4, represented by the Formula IIA 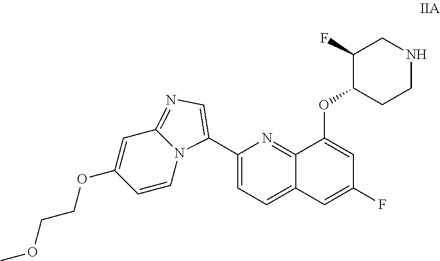
and having the chemical name 6-fluoro-8-((3S,4S)-3-fluoropiperidin-4-yloxy)-2-(7-(2-methoxyethoxy)imidazo[1,2-a]pyridin-3-yl)quinoline.

6. The compound of claim 4, represented by the Formula IIB: 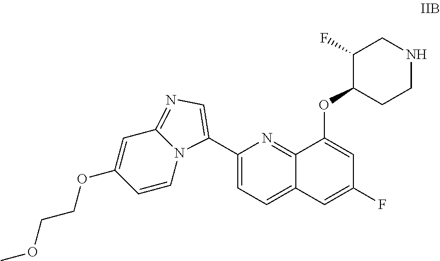
and having the chemical name 6-fluoro-8-((3R,4R)-3-fluoropiperidin-4-yloxy)-2-(7-(2-methoxyethoxy)imidazo[1,2-a]pyridin-3-yl)quinoline.

7. A pharmaceutical composition, which comprises a compound as defined in claim 1, or a pharmaceutically acceptable salt thereof, and a pharmaceutically acceptable diluent or carrier.

8. A method of treating cancer in a mammal, which comprises administering to said mammal a therapeutically effective amount of a compound as defined in claim 1, or a pharmaceutically acceptable salt thereof.

9. A method of treating fibrosis in a mammal, which comprises administering to said mammal a therapeutically effective amount of a compound as defined in claim 1, or a pharmaceutically acceptable salt thereof.

10. A compound as defined in claim 1, or a pharmaceutically acceptable salt thereof for use in the treatment of cancer.

11. A compound as defined in claim 1, or a pharmaceutically acceptable salt thereof for use in the treatment of fibrosis.

12. A process for the preparation a compound of claim 1, which comprises:
coupling a compound having formula III 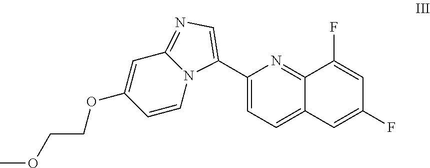
with a compound having formula IV 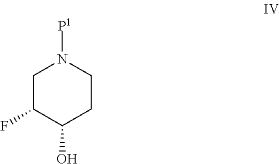
where P¹ is an amine protecting group, in the presence of a catalyst or a base, followed by removing the protecting group and forming a salt, if desired.

13. A process for preparing a compound of claim 4, comprising:
coupling a compound having the formula VIII 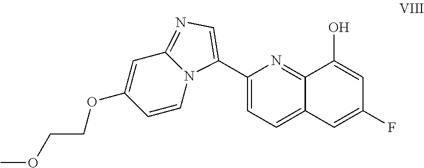
with a compound having the formula IX 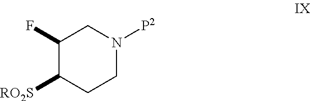
where P² is an amine protecting group and RSO₂ is an alkyl or aryl sulfonate, in the presence of base, followed by removing the protecting group and forming a salt, if desired.
